(19) United States
(12) Patent Application Publication
(10) Pub. No.: US 2015/0018864 A1
(43) Pub. Date: Jan. 15, 2015

(54) Pocket dissector

(71) Applicant: Jay Pensler, Chicago, IL (US)

(72) Inventor: Jay Pensler, Chicago, IL (US)

(21) Appl. No.: 14/170,585

(22) Filed: Feb. 1, 2014

Related U.S. Application Data
(60) Provisional application No. 61/845,181, filed on Jul. 11, 2013.
(60) Provisional application No. 61/901,159, filed on Nov. 7, 2013.

(51) Int. Cl. A61B 17/3201

(52) U.S. Cl. CPC A61B 17/3201
USPC 606/174

(57) ABSTRACT

A shaping tool for forming a surgical pocket in breast surgery may include a handle portion including a pair of opposing handles to open and close the shaping tool, a center portion connected to the handle portion including a shaft to allow the pair of opposing handles to pivot and a shaping portion connected to the center portion to form the pocket. This dissector creates a specific pocket size for a breast implant. The dissector is placed through a skin incision then is opened in a scissor like action to dissect the precise pocket for breast implant placement.

### PRIORITY

The present application claims priority under 35 USC section 119 based on the first provisional application filed on Jul. 11, 2013 with a Ser. No. of 61/845,181 and a second provisional application filed on Nov. 11, 2013 with the Ser. No. of 61/901,159.

### FIELD OF THE INVENTION

The present invention relates to tools and more particularly to a tool to dissect a precise cavity for placement of a breast implant

### BACKGROUND

A large number of women undergo elective surgery in order to enhance the size and shape of their breasts by having breast implants placed. In addition reconstructive surgery for breast cancer may necessitate the placement of a breast implant. During such procedures, especially for a procedure with placement of anatomically shaped breast implant, it is necessary to dissect a precisely defined space under the existing breast in order to provide a new cavity sufficiently large to accommodate the breast implant.

### SUMMARY

This device is a dissector which creates a specific pocket size for a specific implant. The invention is placed through a skin incision by the surgeon in the dissection position for the device. The device can only be and is fully opened to a precise, predetermined point regulated by mechanical stops on the device.

The device is placed like a partially opened scissor into the breast area incision in the appropriate tissue plane prior to implant placement and dissects the tissue as the device is gently pushed further into the body. The device is then opened to a predesigned endpoint that is manufactured into the device in a scissor like movement. Once the device is fully opened a mechanical stop prevents the device from opening further. The device is then closed and removed from the patient and the implant which corresponds to the device is placed into the patient. The skin incision and the various tissues layers are closed. In a reconstructive case several devices may be placed into the defect, smaller to larger, and the defect accessed for the appropriate implant. The appropriate implant is determined by the device which most completely opens into the shape of the defect in reconstructive cases. This device minimizes the likelihood in reconstructive cases of too small an implant being chosen and in cosmetic cases for too large a pocket being created.

The handle may be substantially rectangular or round.

The center portion may include a concave edge to enhance dissection of tissue.

The concave edge may include a serrated edge to further enhance dissection of the soft tissues.

The shaping portion may include a convex edge.

The convex edge may include a serrated edge.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a side view of an open shaping tool of the present invention;

FIG. 2 illustrates a side view of a closed shaping tool of the present invention;

FIG. 3 illustrates an opposing side view of the open shaping tool of the present invention;

FIG. 4 illustrates the shaping tool of the present invention being held by a user;

FIG. 5 illustrates a top view of the shaping tool of the present invention;

FIG. 6 illustrates a bottom view of the shaping tool of the present invention;

FIG. 7 is a perspective view of the invention shown in closed configuration;

FIG. 8 is a perspective view of the invention shown in open configuration;

FIG. 9 is a section view of the invention section view of the invention along line 3-3 in FIG. 7;

FIG. 10 is a section view of the invention section view of the invention along line 4-4 in FIG. 8;

FIGS. 11-13 are a section detail of the invention shown in use (pre-insertion) through exemplary tissue 20.

### DETAILED DESCRIPTION

This invention is a surgical tool which creates a specific pocket size for the placement of a breast implant. The invention is placed through a skin incision then is opened in a scissor like action to dissect a precise pocket for breast implant placement.

This newly dissected surgical pocket should be sufficiently large to accommodate the desired breast implant and also should not be too large to leave a gap between the breast implant and the surrounding tissues. This is a particularly crucial aspect of breast implant surgery when using a shaped breast implant. A shaped breast implant is an implant with the shape of a teardrop. The implant slopes to a higher and fuller projection towards the bottom of the implant. The asymmetric shape of the implant requires that a precise pocket be dissected for the implant which must fit the implant like a glove to minimize the possibility of implant rotation in the patient after placement. If a shaped implant rotates a substandard result will ensue typically necessitating an additional surgery to correct the problem. Also a pocket that is not precise may result in folding of the implant and the perception of rippling which is an undesirable result. There is no product prior to this invention which will dissect precisely a pre-designated pocket size for specific breast implants.

In reconstructive surgery after mastectomy, a unilateral defect is created which will require placement of an implant, often a shaped implant. It is critical to determine the correct implant size to accommodate the surgical defect. If too small a shaped implant is used for the defect a similar situation to the case in elective breast augmentation will occur with rotation of the implant after surgical placement with similar consequences typically requiring a surgery to correct the problem. The described device may be utilized to measure the defect after reconstructive surgery with resection of tissue to eradicate disease and facilitate correct selection of the appropriate implant size require to precisely fill the defect. The sizing of the defect would be accomplished by opening the device when placed into the defect at surgery.

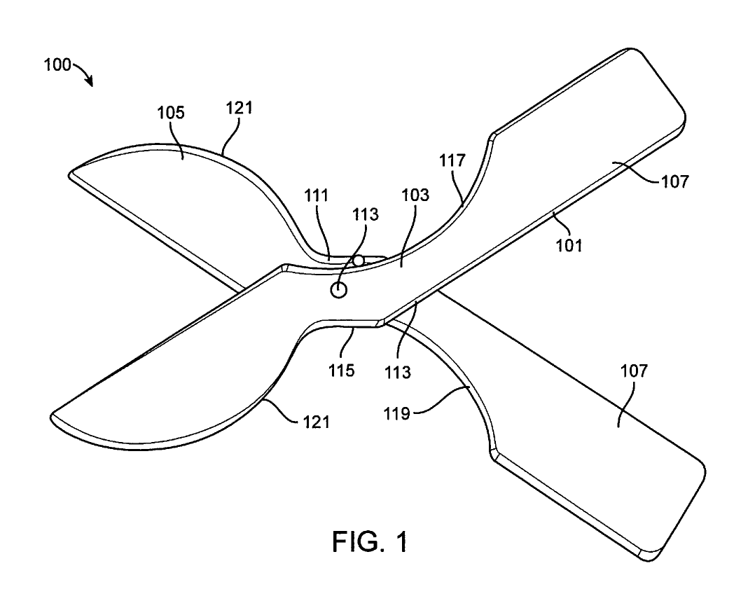

FIG. 1 illustrates the shaping tool 100 of the present invention in an open position, and the shaping tool 100 may include a handle portion 101 to allow the user to manipulate the shaping tool 100, a center portion 103 to connect the handle portion 101 to the shaping portion 105 and the shaping portion 105 to shape a cavity or surgical pocket for a suitable implant (not shown) which may be a breast implant to be inserted into the breast area of a patient. The handle portion 101, the center portion 103 and the shaping portion 105 may be the substantially same thickness which may be a relatively narrow flat wall and which may be substantially rigid. The handle portion 101, the center portion 103 and the shaping portion 105 may be an integral tool (a single piece) which may be formed from metal, plastic or other appropriate material.

The handle portion 101 may include a pair of opposing handles 107 which may be substantially rectangular shaped (other shapes are within the scope of the invention), which may be substantially mirrored handles 107 and pivot to open and close an opposing pair of shaping arms 109 which may be a truncated dome shaped arm.

The center portion 103 may include a pair of opposed and mirror center arms 111 and which may be pivotably connected by a shaft 113. The center portion 103 may include a straight edge 113 which may extend from the handle 107, may include an inclined edge 115 which may be connected to the straight edge 113, and may include a concave edge 117. The concave edge 117 may include a serrated edge 119 on a portion or the entire concave edge 117 and may extend from the handle portion 101 to the shaping portion 105.

The shaping portion 105 may be a truncated dome shape and may include a convex edge 121 and a substantially straight edge 123 which may extend from the concave edge 117 to the convex edge 121. The convex edge 121 may extend from the inclined edge 115 to the straight edge 123 and may include a serrated edge which may extend over a portion or the entire edge, and the straight edge 123 may include a serrated edge which may extend over a portion or the entire edge.

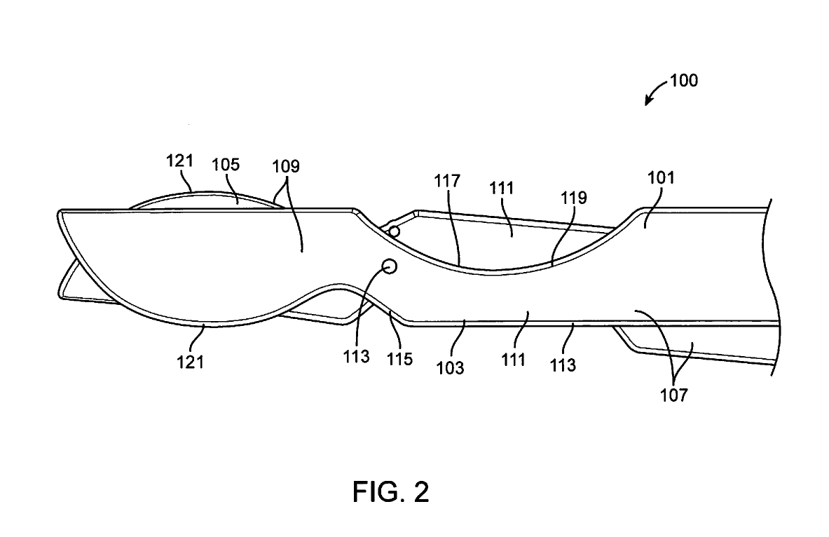

FIG. 2 illustrates the shaping tool 100 of the present invention in a closed position, and the shaping tool 100 may include a handle portion 101 to allow the user to manipulate the shaping tool 100, a center portion 103 to connect the handle portion 101 to the shaping portion 105 and the shaping portion 105 to shape a cavity for a suitable implant (not shown) which may be a breast implant to be inserted into the breast area of a patient. The handle portion 101, the center portion 103 and the shaping portion 105 may be the substantially same thickness which may be relatively narrow wall and which may be substantially rigid.

The handle portion 101 may include a pair of opposing handles 107 which may be substantially rectangular shaped (other shapes are within the scope of the invention), which may be substantially mirrored handles 107 and pivot to open and close an opposing pair of shaping arms 109 which may be a truncated dome shaped arm.

The center portion 103 may include a pair of opposed and mirror center arms 111 and which may be pivotably connected by a shaft 113. The center portion 103 may include a straight edge 113 which may extend from the handle 107, may include an inclined edge 115 which may be connected to the straight edge 113, and may include a concave edge 117. The concave edge 117 may include a serrated edge 119 on a portion or the entire concave edge 117 and may extend from the handle portion 101 to the shaping portion 105.

The shaping portion 105 may be a truncated dome shape and may include a convex edge 121 and a substantially straight edge 123 which may extend from the concave edge 117 to the convex edge 121. The convex edge 121 may extend from the inclined edge 115 to the straight edge 123 and may include a serrated edge which may extend over a portion or the entire edge, and the straight edge 123 may include a serrated edge which may extend over a portion or the entire edge.

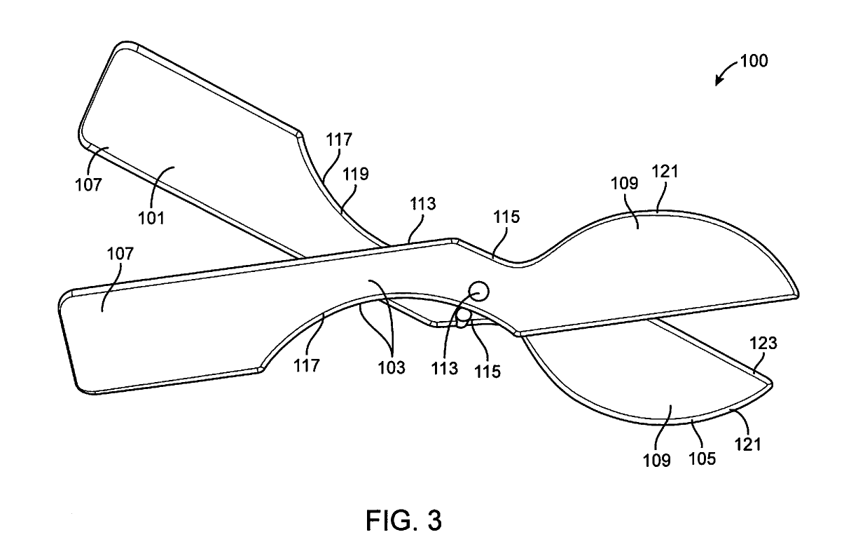

FIG. 3 illustrates the shaping tool 100 (the opposing side of FIG. 1) of the present invention in an open position, and the shaping tool 100 may include a handle portion 101 to allow the user to manipulate the shaping tool 100, a center portion 103 to connect the handle portion 101 to the shaping portion 105 and the shaping portion 105 to shape a cavity for a suitable implant (not shown) which may be a breast implant to be inserted into the breast area of a patient. The handle portion 101, the center portion 103 and the shaping portion 105 may be the substantially same thickness which may be relatively narrow wall and which may be substantially rigid.

The handle portion 101 may include a pair of opposing handles 107 which may be substantially rectangular shaped (other shapes are within the scope of the invention), which may be substantially mirrored handles 107 and pivot to open and close an opposing pair of shaping arms 109 which may be a truncated dome shaped arm.

The center portion 103 may include a pair of opposed and mirror center arms 111 and which may be pivotably connected by a shaft 113. The center portion 103 may include a straight edge 113 which may extend from the handle 107, may include an inclined edge 115 which may be connected to the straight edge 113, and may include a concave edge 117. The concave edge 117 may include a serrated edge 119 on a portion or the entire concave edge 117 and may extend from the handle portion 101 to the shaping portion 105.

The shaping portion 105 may be a truncated dome shape and may include a convex edge 121 and a substantially straight edge 123 which may extend from the concave edge 117 to the convex edge 121. The convex edge 121 may extend from the inclined edge 115 to the straight edge 123 and may include a serrated edge which may extend over a portion or the entire edge, and the straight edge 123 may include a serrated edge which may extend over a portion or the entire edge.

FIG. 4 illustrates the shaping tool 100 of the present invention being held by a user and illustrates the handle portion 101, the center portion 103 and the shaping portion 105. Note the

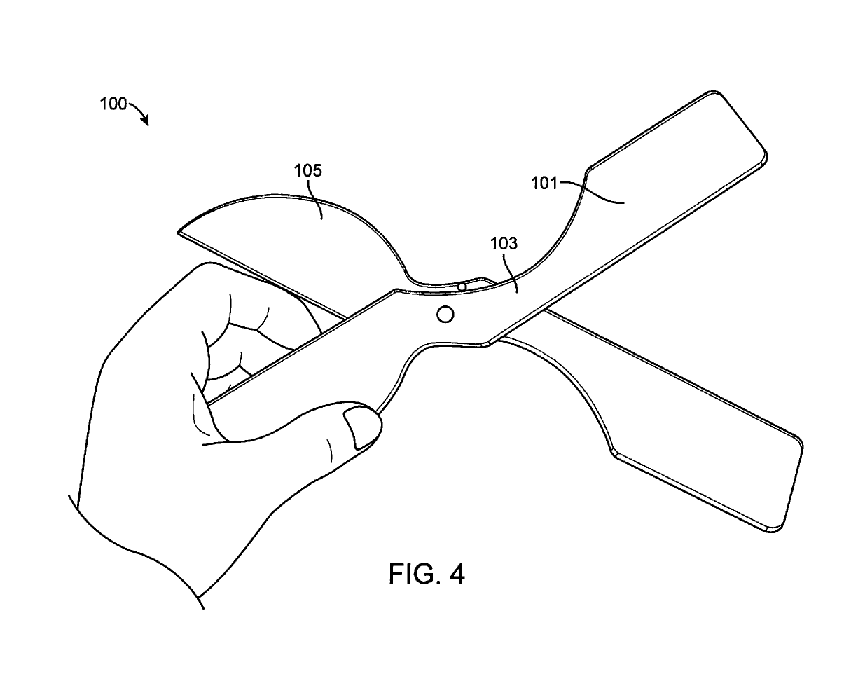

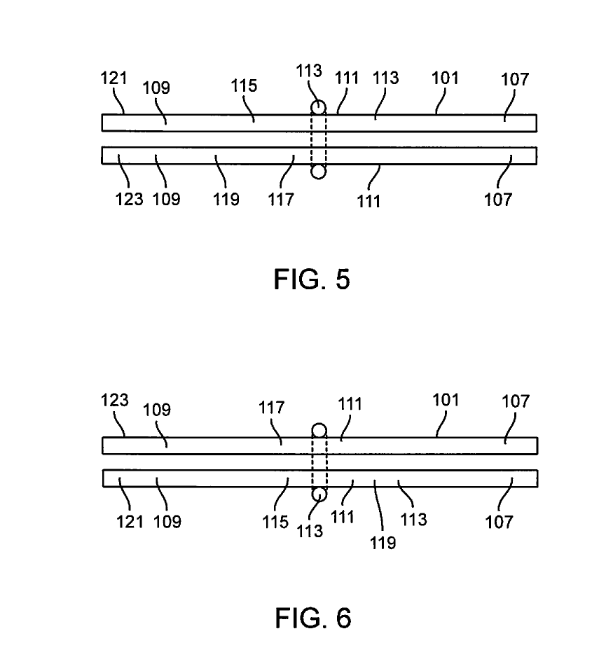

FIGS. 5 and 6 illustrate the top and bottom view respectively of the shaping tool 100 of the present invention, and the shaping tool 100 may include a handle portion 101 to allow the user to manipulate the shaping tool 100, a center portion 103 to connect the handle portion 101 to the shaping portion 105 and the shaping portion 105 to shape a cavity for a suitable implant (not shown) which may be a breast implant to be inserted into the breast area of a patient. The handle portion 101, the center portion 103 and the shaping portion 105 may be the substantially same thickness which may be relatively narrow wall and which may be substantially rigid.

The handle portion 101 may include a pair of opposing handles 107 which may be substantially rectangular shaped (other shapes are within the scope of the invention), which may be substantially mirrored handles 107 and pivot to open and close an opposing pair of shaping arms 109 which may be a truncated dome shaped arm.

The center portion 103 may include a pair of opposed and mirror center arms 111 and which may be pivotably connected by a shaft 113. The center portion 103 may include a straight edge 113 which may extend from the handle 107, may include an inclined edge 115 which may be connected to the straight edge 113, and may include a concave edge 117. The concave edge 117 may include a serrated edge 119 on a portion or the entire concave edge 117 and may extend from the handle portion 101 to the shaping portion 105.

The shaping portion 105 may be a truncated dome shape and may include a convex edge 121 and a substantially straight edge 123 which may extend from the concave edge 117 to the convex edge 121. The convex edge 121 may extend from the inclined edge 115 to the straight edge 123 and may include a serrated edge which may extend over a portion or the entire edge, and the straight edge 123 may include a serrated edge which may extend over a portion or the entire edge.

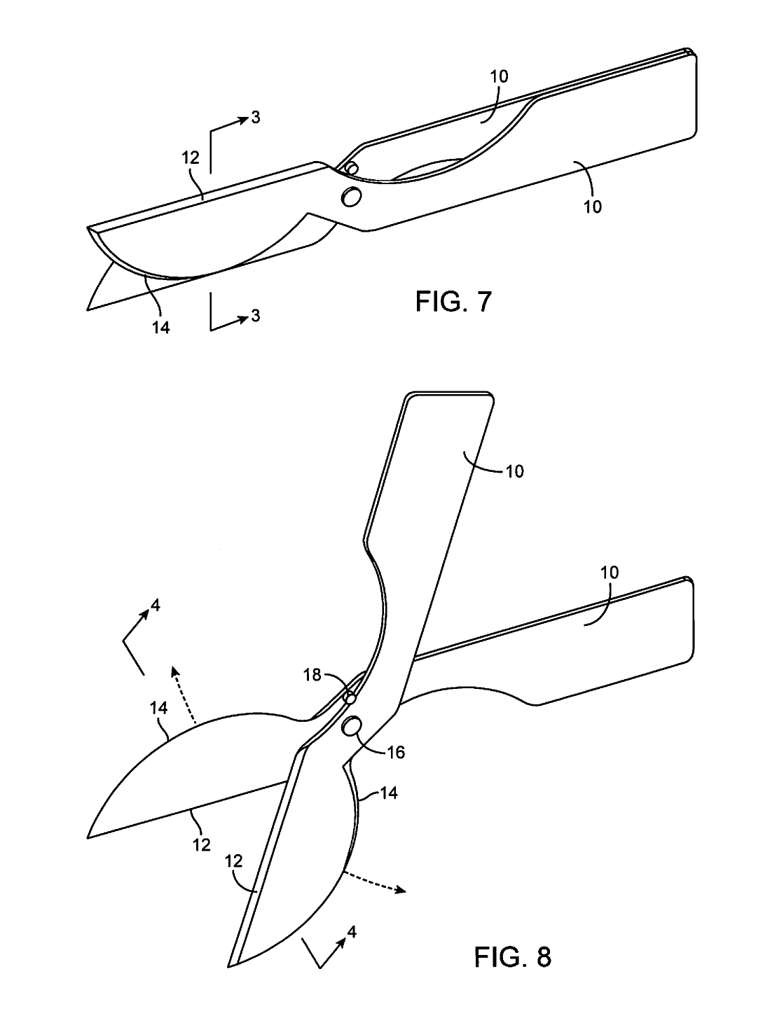

FIGS. 7-10 illustrate an alternate embodiment of the pocket dissector tool of the present invention.

A pair of opposing handles 10 are shown in FIG. 7 positioned substantially adjacent while the tool is in a closed position, and the opposing handles.

One of the handles 10 may include a stop 18 (an inward extending arm) to limit the movement of the handles 10 by engaging the stop 18 with the curved edge of the opposing handle 10. A straight cutting edge 12 is additionally illustrated along with a curve cutting edge 14. The handles 10 may pivot by the pivot (device) 16.

FIG. 8 illustrates the handles 10 in an open position and engaging the stop 18 by engaging the opposing handle 10.

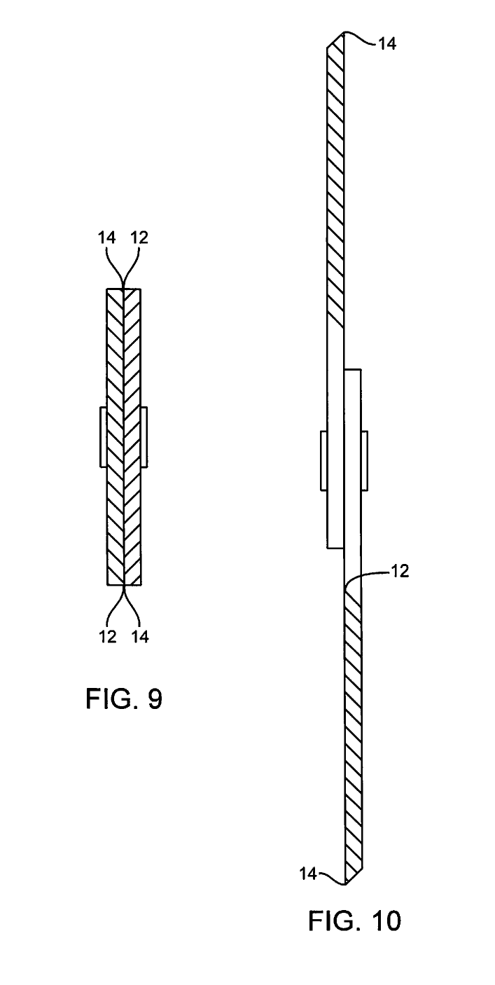

FIG. 9 illustrates a section view of the invention along line 3-3 as illustrated in FIG. 7.

FIG. 10 illustrates a section view of the invention along line 4-4 as illustrated in FIG. 8.

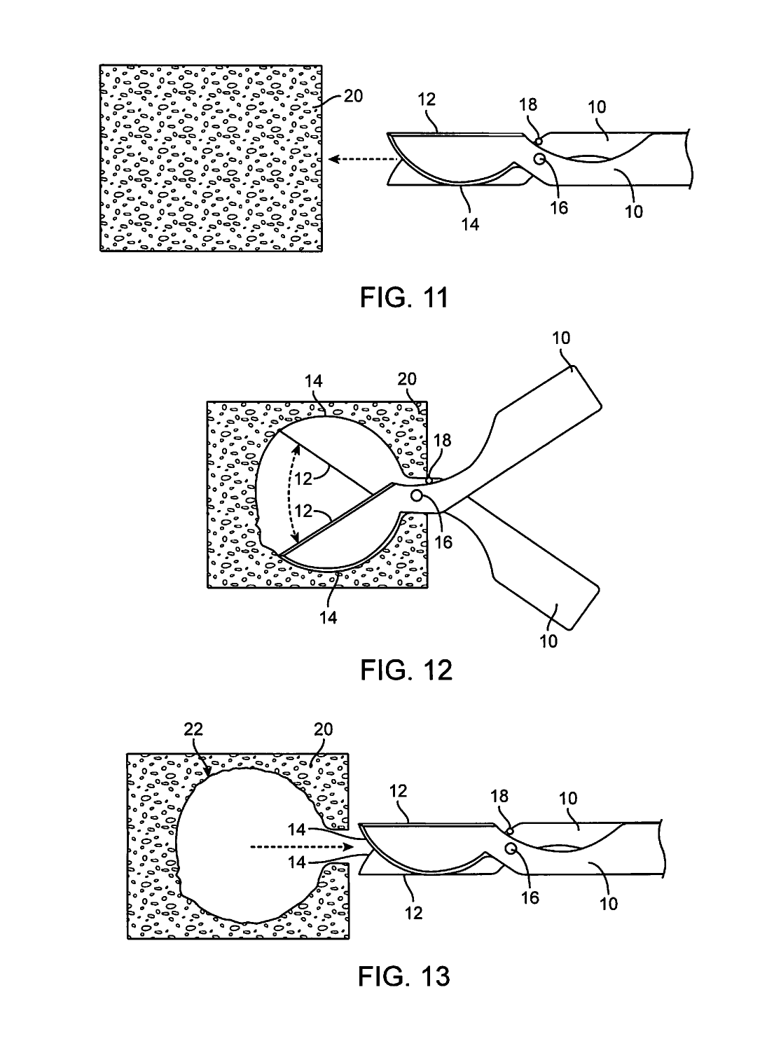

FIG. 11 illustrates the tool of the present invention in a closed position before being inserted into the tissue 20.

FIG. 12 illustrates the tool of the present invention engaging the tissue 20 and being opened in order to form a pocket 22 (a cavity within the tissue)

FIG. 13 illustrates the tool of the present invention being withdrawn from the tissue 20 in a closed position, leaving a pocket 22 (or cavity) within the tissue 20.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A shaping tool for forming a pocket in breast surgey, comprising:
a handle portion including a pair of opposing handles to open and close the shaping tool;
a center portion connected to the handle portion including a shaft to allow the pair of opposing handles to pivot;
a shaping portion connected to the center portion to form the pocket;
wherein the shaping portion be shaped as a truncated dome.

2) A shaping tool for forming a pocket as in claim 1, wherein the handle is substantially rectangular.

3) A shaping tool for forming a pocket as in claim 1, wherein the center portion includes a concave edge.

4) A shaping tool for forming a pocket as in claim 3, wherein the concave edge includes a serrated edge.

5) A shaping tool for forming a pocket as in claim 1, wherein the shaping portion includes a convex edge.

6) A shaping tool for forming a pocket as in claim 5, wherein the convex edge includes a serrated edge.
